SEQUENCE LISTING (26 sequences)

SEQ ID NO: 1 - protein, 347 aa, Homo sapiens
MASGILVNVKEEVTCPICLELLTQPLSLDCGHSFCQACLTANHKKSMLDKGESSCPVCRISYQPENIRPNRHVANIVEKLREVKLSPEGQKVDHCARHGEKLLLFCQEDGKVICWLCERSQEHRGHHTFLTEEVAREYQVKLQAALEMLRQKQQEAEELEADIREEKASWKTQIQYDKTNVLADFEQLRDILDWEESNELQNLEKEEEDILKSLTNSETEMVQQTQSLRELISDLEHRLQGSVMELLQGVDGVIKRTENVTLKKPETFPKNQRRVFRAPDLKGMLEVFRELTDVRRYWGKEKSHYHKPPCGLSLLLSLSFRILCSLLGSCFKIYDSPSKTHITYPSL

SEQ ID NO: 2 - protein, 9 aa, Homo sapiens
ASGILVNVK

SEQ ID NO: 3 - protein, 6 aa, Homo sapiens
KSMLDK

SEQ ID NO: 4 - protein, 14 aa, Homo sapiens
SMLDKGESSCPVCR

SEQ ID NO: 5 - protein, 9 aa, Homo sapiens
GESSCPVCR

SEQ ID NO: 6 - protein, 8 aa, Homo sapiens
HVANIVEK

SEQ ID NO: 7 - protein, 7 aa, Homo sapiens
LSPEGQK

SEQ ID NO: 8 - protein, 18 aa, Homo sapiens
LLLFCQEDGKVICWLCER

SEQ ID NO: 9 - protein, 8 aa, Homo sapiens
VICWLCER

SEQ ID NO: 10 - protein, 14 aa, Homo sapiens
EYQVKLQAALEMLR

SEQ ID NO: 11 - protein, 11 aa, Homo sapiens
LQAALEMLRQK

SEQ ID NO: 12 - protein, 7 aa, Homo sapiens
EEKASWK

SEQ ID NO: 13 - protein, 7 aa, Homo sapiens
TQIQYDK

SEQ ID NO: 14 - protein, 16 aa, Homo sapiens
DILDWEESNELQNLEK

SEQ ID NO: 15 - protein, 7 aa, Homo sapiens
EEEDILK

SEQ ID NO: 16 - protein, 9 aa, Homo sapiens
ELISDLEHR

SEQ ID NO: 17 - protein, 18 aa, Homo sapiens
LQGSVMELLQGVDGVIKR

SEQ ID NO: 18 - protein, 8 aa, Homo sapiens
RTENVTLK

SEQ ID NO: 19 - protein, 7 aa, Homo sapiens
KPETFPK

SEQ ID NO: 20 - protein, 10 aa, Homo sapiens
KPETFPKNQR

SEQ ID NO: 21 - protein, 8 aa, Homo sapiens
VFRAPDLK

SEQ ID NO: 22 - protein, 7 aa, Homo sapiens
GMLEVFR

SEQ ID NO: 23 - protein, 7 aa, Homo sapiens
ELTDVRR

SEQ ID NO: 24 - protein, 6 aa, Homo sapiens
YWGKEK

SEQ ID NO: 25 - protein, 7 aa, Homo sapiens
IYDSPSK

SEQ ID NO: 26 - protein, 8 aa, Homo sapiens
THITYPSL

(19) United States
(12) Patent Application Publication
(10) Pub. No.: US 2006/0115867 A1
(43) Pub. Date: Jun. 1, 2006

(54) TRIM 5 related protein as a biomarker of neurodegenerative disease

(75) Inventors: Ira L. Goldknopf, The Woodlands, TX (US); Essam A. Sheta, The Woodlands, TX (US); Stanley H. Appel, Houston, TX (US); Ericka P. Simpson, Pearland, TX (US); Albert A. Yen, Pearland, TX (US)

Correspondence Address: ELIZABETH R. HALL, 1722 MARYLAND STREET, HOUSTON, TX, 77006, US

(73) Assignee: Power3 Medical Products, Inc., The Woodlands, TX (US)

(21) Appl. No.: 11/039,600

(22) Filed: Jan. 19, 2005

Related U.S. Application Data
(60) Provisional application No. 60/632,217, filed on Dec. 1, 2004.

(51) Int. Cl. C07K 14/705; C07K 16/28; G01N 33/53; G01N 33/537; G01N 33/543

(52) U.S. Cl. 435/7.93; 530/350; 530/388.22

(57) ABSTRACT

The present invention relates to a biomarker for neurodegenerative disease, including amyotrophic lateral sclerosis (ALS), Alzheimer's (AD), and Parkinson's (PD) disease. More particularly, the present invention relates to the identification of a tripartite motif protein TRIM5 isoform gamma related protein as a biomarker useful for the detection, diagnosis, and differentiation of neurodegenerative disease, including but not limited to ALS, AD, and PD.

### CROSS REFERENCE TO RELATED APPLICATIONS

This application claims priority to U.S. Provisional Patent Application Ser. No. 60/632,217 filed Dec. 1, 2004 and entitled “A TRIM 5 Related Protein as a Biomarker of Neurodegenerative Disease” by inventors Ira L. Goldknopf, et al.

### BACKGROUND OF THE INVENTION

1. Field of the Invention

The invention relates to the identification of a biomarker for the detection of neurodegenerative disease. More particularly, the present invention relates to the identification of a tripartite motif protein TRIM5 isoform gamma related protein as a biomarker useful in the diagnosis of amyotrophic lateral sclerosis (ALS), Alzheimer's (AD), and Parkinson's (PD) disease.

2. Description of the Related Art

Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of certain proteins already known to be involved in certain biological functions has progressed from research applications alone to use in disease screening and diagnosis for clinicians. However, proteonomic testing for diagnostic purposes remains in its infancy. There is, however, a great deal of interest in using proteomics for the elucidation of potential disease biomarkers.

Detection of abnormalities in the genome of an individual can reveal the risk or potential risk for individuals to develop a disease. The transition from risk to emergence of disease can be characterized as an expression of genomic abnormalities in the proteome. Thus, the appearance of abnormalities in the proteome signals the beginning of the process of cascading effects that can result in the deterioration of the health of the patient. Therefore, detection of proteomic abnormalities at an early stage is desirable in order to allow for detection of disease either before it is established or in its earliest stages where treatment may be effective.

Recent progress using a novel form of mass spectrometry called surface enhanced laser desorption and ionization time of flight (SELDI-TOF) for the testing of ovarian cancer has led to an increased interest in proteomics as a diagnostic tool (Petrocoin, E. F. et al. 2002. Lancet 359:572-577). Furthermore, proteomics has been applied to the study of breast cancer through use of 2D gel electrophoresis and image analysis to study the development and progression of breast carcinoma in patients (Kuerer, H. M. et al. 2002. Cancer 95:2276-2282). In the case of breast cancer, breast ductal fluid specimens were used to identify distinct protein expression patterns in bilateral matched pair ductal fluid samples of women with unilateral invasive breast carcinoma.

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to raise protective antibodies or in a kit to identify the presence or absence of the biomarker in individuals who may have been exposed to mustard poisoning. U.S. Pat. No. 6,326,209 B1 discloses measurement of total urinary 17 ketosteroid-sulfates as biomarkers of biological age. U.S. Pat. No. 6,693,177 B1 discloses a process for preparation of a single biomarker specific for O-acetylated sialic acid and useful for diagnosis and outcome monitoring in patients with lymphoblastic leukemia.

Neurodegenerative diseases are difficult to diagnose, particularly in their earlier stages, as currently there are no biomarkers available for either the early diagnosis or treatment of neurodegenerative diseases such as amyotrophic lateral sclerosis (ALS), Alzheimer's (AD), or Parkinson's (PD) disease.

Therefore, there remains a need for better ways to detect, diagnose, and distinguish ALS and other neurodegenerative diseases, including a need for specific biomarkers of neurodegenerative disease.

### SUMMARY OF THE INVENTION

The present invention relates to the tripartite motif protein TRIM5 isoform gamma (TRIM5) and related proteins and peptides as biomarkers for neurodegenerative disease, where an increase in the concentration of tripartite motif protein TRIM5 isoform gamma and related proteins is an indicator of neurodegenerative disease.

One aspect of the present invention is a method for screening for neurodegenerative disease comprising: obtaining a serum sample from a test subject; determining the quantity of at least one tripartite motif protein TRIM5 isoform gamma related peptide in the serum sample; and comparing the quantity of the tripartite motif protein TRIM5 isoform gamma related peptide in the test subject serum sample with a range of normal value a of the tripartite motif protein TRIM5 isoform gamma related peptide in control subjects; whereby an increase in the quantity of the tripartite motif protein TRIM5 isoform gamma related protein in the serum sample to a level greater than the range of normal values of the tripartite motif protein TRIM5 isoform gamma related peptide is indicative of a neurodegenerative condition.

Another aspect of the present invention is a method of diagnosing a neurodegenerative disease comprising: collecting a serum sample from a test subject; analyzing the serum sample for an increased expression of tripartite motif protein TRIM5 isoform gamma related protein; and using the expression of tripartite motif protein TRIM5 isoform gamma related protein to diagnose the test subject.

Still another aspect of the present invention is a method for diagnosing neurodegenerative disease comprising: obtaining a serum sample from a patient and a set of control serum samples; determining the quantity of a tripartite motif protein TRIM5 isoform gamma related peptide in the patient serum sample and the set of control samples; and comparing the quantity of the tripartite motif protein TRIM5 isoform gamma related protein in the patient serum with the quantity of the tripartite motif protein TRIM5 isoform gamma related peptide in the set of control samples to diagnose a neurodegenerative condition.

Yet another aspect of the present invention is a method for diagnosing neurodegenerative disease comprising: obtaining a patient serum sample; determining a protein expression pattern of the serum sample by two-dimensional gel electrophoresis; quantitating a tripartite motif protein TRIM5 isoform gamma protein related protein in the protein expression pattern; and using the quantity of the tripartite motif protein TRIM5 isoform gamma related protein to diagnose a neurodegenerative condition.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates the differentially expressed proteins visualized in a gel overlay of a 2D gel of control serum and a 2D gel of serum collected from an ALS patient.

### DESCRIPTION OF THE PREFERRED EMBODIMENTS

The present invention relates to a biomarker for neurodegenerative disease, including amyotrophic lateral sclerosis (ALS), Alzheimer's (AD), and Parkinson's (PD) disease. More particularly, the present invention relates to the identification of a tripartite motif protein TRIM5 isoform gamma related protein (TRIM5) as a biomarker useful for the detection, diagnosis, and differentiation of neurodegenerative disease, including but not limited to ALS, AD, and PD.

The method for identification of the tripartite motif protein TRIM5 isoform gamma related protein as a biomarker for neurodegenerative disease is based on the comparison of 2D gel electrophoretic images of serum obtained from human subjects with and without diagnosed neurodegenerative disease.

2D gel electrophoresis has been used in research laboratories for biomarker discovery since the 1970's (Goldknopf, I. L. et al. 1977. Proc. Natl. Acad. Sci. USA 74:864-868). In the past, this method has been considered highly specialized, labor intensive and non-reproducible. Only recently with the advent of integrated supplies, robotics, and software combined with bioinformatics has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as phosphorylation, ubiquitination, conjugation with ubiquitin-like proteins, acetylation, and glycosylation. These are important variables in cell regulatory processes involved in cancer and other diseases.

There are few comparable alternatives to 2D gels for tracking changes in protein expression patterns related to disease progression. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between two samples from the same individual or between several individuals.

Proteins of interest can be excised from the gels and the proteins can then be identified by in-gel digestion and matrix assisted laser desorption time of flight mass spectroscopy (MALDI-TOF MS) based peptide mass fingerprinting and database searching, or liquid chromatography with tandem mass spectrometry partial sequencing of individual peptides (LCMS/MS).

The identification of the tripartite motif protein TRIN45 isoform gamma as a biomarker of neurodegenerative disease was based on a comparison of the 2D gel electrophoretic images of serum samples obtained from 24 normal control subjects without any neurodegenerative disease, 92 patients with diagnosed ALS, 36 patients with diagnosed AD, and 26 patients with diagnosed PD.

Sample Collection and Preparation

Sample collection and storage has been performed in many different ways depending on the type of sample and the conditions of the collection process. In the present study, serum samples were collected, aliquoted and stored in a −80° C. freezer before analysis.

In a preferred embodiment of the invention, the serum samples were removed from −80° C. and placed on ice for thawing. To each 10 μl of sample, 90 μl of LB-1 buffer (7M urea, 2M Thiourea, 1% DTT, 1% Triton X-100, 1×Protease inhibitors, and 0.5% Ampholyte pH 3-10) was added and the mixture vortexed. The sample was incubated at room temperature for about 5 minutes.

Two Dimensional-Electrophoresis of Samples

Separation of the proteins in the serum samples was then performed using 2D gel electrophoresis. The 2D gel electrophoretic images were obtained, compared and analyzed as described in the U.S. Provisional Patent Application Ser. No. 60/614,315 entitled “Differential Protein Expression Patterns Related to Disease States” filed Sep. 29, 2004 and incorporated herein by reference.

After the serum samples had been incubated with the LB-1 buffer, 300μl UPPA-I (Perfect Focus, Genotech) was added to each sample and the sample vortexed and incubated on ice for 15 minutes. Next 600 μl UPPA-IL (Perfect Focus, Genotech) was added to each tube, vortexed and centrifuged at about 15,000×g for 5 minutes at 4° C. The entire supernatant was carefully removed by vacuum aspiration. Repeat centrifugation at about 15,000×g for 30 seconds was performed. The remaining supernatant was removed by vacuum aspiration.

The pellet was suspended in 25 μl of ultra pure water and vortexed. Next 1 ml of OrgoSol (Perfect Focus, Genotech, prechilled at −20° C.) and 5 μl SEED (Perfect Focus, Genotech) were added to each pellet and incubated at −20° C. for about 30 minutes. The pellet was suspended using repeated vortexing bursts of about 20-30 seconds each. The tubes were then centrifuged at about 15,000×g for 5 minutes. The entire supernatant was carefully removed by vacuum aspiration. The water suspension and the OrgoSol-SEED wash of the pellet were repeated to yield a protein pellet.

The protein pellet was air dried for about 5 minutes, then the pellet was dissolved in an appropriate amount of isoelectric focusing (IEF) loading buffer (LB-1), incubated at room temperature and vortexed periodically until the pellet was dissolved to visual clarity. The samples were centrifuged briefly before a protein assay was performed on the sample.

Approximately 100 μg of the solubilized protein pellet was suspended in a total volume of 184 μl of IEF loading buffer and 1 μl Bromophenol Blue. Each sample was loaded onto an 11 cm EEF strip (Bio-Rad), pH 5-8, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min at 250V followed by a linear voltage increase to 8000V over 2.5 hours. A final rapid focusing was performed at 8000V until 20,000 volt-hours were achieved. Running the EEF strip at 500V until the strips were removed finished the isoelectric focusing process.

Isoelectric focused strips were incubated on an orbital shaker for 15 min with equilibration buffer (2.5 ml buffer/strip). The equilibration buffer contained 6M urea, 2% SDS, 0.375M HCl, and 20% glycerol, as well as freshly added DTT to a final concentration of 30 mg/ml. An additional 15 min incubation of the EEF strips in the equilibration buffer was performed as before, except freshly added iodoacetamide (C₂H₄INO) was added to a final concentration of 40 mg/ml. The IPG strips were then removed from the tray using clean forceps and washed five times in a graduated cylinder containing the Bio Rad running buffer 1×Tris-Glycine-SDS.

The washed IEF strips were then laid on the surface of Bio Rad pre-cast CRITERION SDS-gels 8-16%. The EEF strips were fixed in place on the gels by applying a low melting agarose. A second dimensional separation was applied at 200V for about one hour. After running, the gels were carefully removed and placed in a clean tray and washed twice for 20 minutes in 100 ml of pre-staining solution containing 10% methanol and 7% acetic acid.

Staining and Analysis of the 2D Gels

Once the 2D gel patterns of the serum samples were obtained, the gels were stained with SYPRO RUBY (Bio-Rad Laboratories) and subjected to fluorescent digital image analysis. The protein patterns of the serum samples were analyzed using PDQUEST (Bio-Rad Laboratories) image analysis software.

The 2D gel patterns of the 24 serum samples collected from normal control subjects that were negative for neurodegenerative disease were compared with each other pursuant to the methodology described in the U.S. Provisional Patent Application Ser. No. 60/614,315 entitled “Differential Protein Expression Patterns Related to Disease States” filed Sep. 29, 2004 and incorporated herein by reference. The 24 normal samples all gave similar 2D gel protein patterns that were compiled in a composite normal protein expression pattern.

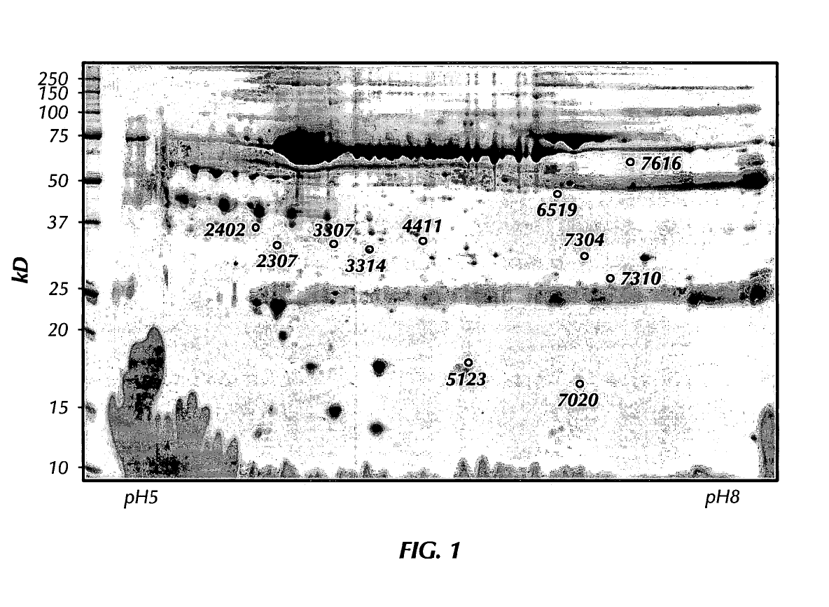

This normal protein expression pattern was then compared to the gel pattern obtained in the 92 ALS patients, the 36 AD patients, and the 26 PD patients. When the gel pattern of an ALS patient was compared to the gel pattern of normal subjects, eleven proteins of particular interest were identified as shown in FIG. 1. One of these protein spots (i.e., spot 2307) was selected for further investigation. Protein 2307 was quantitated by stain intensity in each of the normal (N), ALS, AD and PD serum samples.

To assess the reproducibility of the 2D gels and staining, 75 nanograms of bovine serum albumin (BSA) was run on 9 separate 2D gels. The gels were stained with SYPRO RUBY and the 5 spots that resulted in the BSA region of the gel were then subjected to quantitative analysis using PDQUEST and the Gaussian Peak Value method. The results shown in Table 1 illustrate that the electrophoretic patterns were reproducible and independent of the spot amount over the range tested.

TABLE 1
Reproducibility of Quantitation in 2D Gels - PDQuest
Peak Value of the Major Components of BSA

| | Spot # | | | | |
|---|---|---|---|---|---|
| Replicate # | 9901 | 9902 | 9904 | 9905 | 9906 |
| 1 | 332 | 1152 | 2612 | 739 | 229 |
| 2 | 246 | 974 | 2694 | 513 | 167 |
| 3 | 336 | 1065 | 2354 | 668 | 225 |
| 4 | 311 | 1272 | 3482 | 713 | 198 |
| 5 | 351 | 1168 | 2724 | 733 | 245 |
| 6 | 268 | 1059 | 2753 | 622 | 184 |
| 7 | 452 | 1630 | 4000 | 946 | 281 |
| 8 | 405 | 1195 | 2752 | 870 | 274 |
| 9 | 258 | 1050 | 2716 | 699 | 189 |
| Avg | 329 | 1174 | 2899 | 723 | 221 |
| Stdev | 68 | 193 | 510 | 127 | 40 |
| CV | 21% | 16% | 18% | 18% | 18% |
| ng/spot | 4.4 | 15.6 | 38.6 | 9.6 | 2.9 |

The Isolation and Identification of the Protein 2307

Protein spot 2307 was carefully excised, in-gel digested with trypsin, and subjected to mass fingerprinting analysis by matrix-assisted laser desorption ionization-time of flight mass spectrometry (MALDI-TOF MS) and expert database searching.

Mass spectrometry provides a powerful means of determining the structure and identity of complex organic molecules, including proteins and peptides. The unknown compound is bombarded with high-energy electrons causing it to fragment in a characteristic manner. The fragments, which are of varying weight and charge, are then passed through a magnetic field and separated according to their mass/charge ratios. The resulting characteristic fragmentation pattern of the unknown compound is used to identify and quantitate the unknown compound.

MALDI-TOF MS is a type of mass spectrometry in which the analyte substance is distributed in a matrix before laser desorption. The analyte, co-crystallized with a matrix compound, is subjected to pulse UV laser radiation. The matrix, by strongly absorbing the laser light energy, indirectly causes the analyte to vaporize. The matrix also serves as a proton donor and receptor, acting to ionize the analyte in both positive and negative ionization modes. A protein can often be unambiguously identified by a MALDI-TOF MS analysis of its constituent peptides (produced by either chemical or enzymatic treatment of the sample).

Following differential expression analysis, protein 2307 was carefully excised from the gel for identification. Excised gel spots of protein 2307 were destained by washing the gel spots twice in 100 mM NH₄HCO₃ buffer, followed by soaking the gel spots in 100% acetonitrile for 10 minutes. The acetonitrile was aspirated before adding the trypsin solution.

Typically, a small volume of trypsin solution (approximately 5-15 μg/ml trypsin) is added to the destained gel spots and incubated at 3 hours at 37° C. or overnight at 30° C. The digested peptides were extracted, washed, desalted and concentrated before spotting the peptide samples onto the MALDI-TOF MS target.

Mass spectral analyses of the digested peptides were performed to identify protein 2307. Those of skill in the art are familiar with mass spectral analysis of digested peptides. The mass spectral analysis was conducted on a MALDI-TOF Voyager DE STR (Applied Biosystems). Spectra were carefully scrutinized for acceptable signal-to-noise ratio (S/N) to eliminate spurious artifact peaks from the peptide molecular weight lists.

Both internal and external standards were employed to calibrate any shift in mass values during mass spectroscopic analysis. The external standards were a set of proteins having known molecular weights and known mass/charge ratios in their mass spectrum. A mixture of external standards is placed on the mass spec chip well next to the well that includes an unknown sample. Internal standards are characteristic peaks in the sample spectrum that belong to peptides of the proteolytic enzyme (e.g., trypsin) used to digest the protein spots and extracted along with the digested peptides. Those peaks are used for internal calibration of any deviation of the spectral peaks of the sample.

Corrected molecular weight lists were then subjected to public database searches. The GenBank and dbEST databases maintained by the National Center for Biotechnology Information (hereinafter referred to as the NCBI database) were searched, as well as the SwissProt or Swiss Protein database maintained by ExPasy. Those of skill in the art are familiar with searching databases like the NCBI and SwissProt databases.

The NCBI database search results were displayed according the MOWSE score (a measure of the match probability between the search entries and any proteins identified from the search results). The best match identified by the NCBI database search was the tripartite motif protein TRIM5 isoform gamma (Accession #18204217) having the following sequence:

| (SEQ ID NO:1) | |
|---|---|
| 1 | MASGILVNVK EEVTCPICLE LLTQPLSLDC GHSFCQACLT ANHKKSMLDK GESSCPVCRI |
| 61 | SYQPENIRPN RHVANIVEKL REVKLSPEGQ KVDHCARHGE KLLLFCQEDG KVICWLCERS |
| 121 | QEHRGHHTFL TEEVAREYQV KLQAALEMLR QKQQEAEELE ADIREEKASW KTQIQYDKTN |
| 181 | VLADFEQLRD ILDWEESNEL QNLEKEEEDI LKSLTNSETE MVQQTQSLRE LISDLEHRLQ |
| 241 | GSVMELLQGV DGVIKRTENV TLKKPETFPK NQRRVFRAPD LKGMLEVFRE LTDVRRYWGK |
| 301 | EKSHYHKPPC GLSLLLSLSF RILCSLLGSC FKIYDSPSKT HITYPSL. |

The best match for the protein in gel spot 2307 was TRIM5 with a MOWSE score of 5.79×10⁶ with 43 masses submitted matching the tripartite motif protein TRIM5 isoform gamma. Predominant matched masses included the following sequences.

| | |
|---|---|
| ASGILVNVK | (SEQ ID NO: 2) |
| KSMLDK | (SEQ ID NO: 3) |
| SMLDKGESSCPVCR | (SEQ ID NO: 4) |
| GESSCPVCR | (SEQ ID NO: 5) |
| HVANIVEK | (SEQ ID NO: 6) |
| LSPEGQK | (SEQ ID NO: 7) |
| LLLFCQEDGKVICWLCER | (SEQ ID NO: 8) |
| VICWLCER | (SEQ ID NO: 9) |
| EYQVKLQAALEMLR | (SEQ ID NO: 10) |
| LQAALEMLRQK | (SEQ ID NO: 11) |
| EEKASWK | (SEQ ID NO: 12) |
| TQIQYDK | (SEQ ID NO: 13) |
| DILDWEESNELQNLEK | (SEQ ID NO: 14) |
| EEEDILK | (SEQ ID NO: 15) |
| ELISDLEHR | (SEQ ID NO: 16) |
| LQGSVMELLQGVDGVIKR | (SEQ ID NO: 17) |
| RTENVTLK | (SEQ ID NO: 18) |
| KPETFPK | (SEQ ID NO: 19) |
| KPETFPKNQR | (SEQ ID NO: 20) |
| VFRAPDLK | (SEQ ID NO: 21) |
| GMLEVFR | (SEQ ID NO: 22) |
| ELTDVRR | (SEQ ID NO: 23) |
| YWGKEK | (SEQ ID NO: 24) |
| IYDSPSK | (SEQ ID NO: 25) |
| THITYPSL | (SEQ ID NO: 26) |

Thus, protein 2307 was identified as a tripartite motif protein TRIM5 isoform gamma and/or a closely related protein sharing common peptide sequences such as SEQ ID NOS: 2-26.

Protein 2307 in Normal Subjects and Patients Diagnosed with Neurodegenerative Disease

The protein 2307 concentration was determined in 24 normal subjects, 92 ALS patients, 36 AD patients, and 26 PD patients by quantitating the staining of the synonymous 2D gel protein spot in the 2D gel electrophoresis pattern of each of the serum samples.

Normal serum ranged from an undetectable level of protein 2307 to about 545 ppm, with a mean value of 90.3±71.6 S.E. ppm. The concentration of protein 2307 in the neurodegenerative patients was as follows: the mean concentration of protein 2307 in the 92 ALS patients was 352.9±36.6 S.E. ppm; the mean concentration of protein 2307 in the 36 AD patients was 585.6±58.5 S.E. ppm; and the mean concentration of protein 2307 in the 26 PD patients was 629.2±68.8 S.E. ppm, as shown in Table 2.

TABLE 2

| Diagnosis | # of Patients | Range | Mean Value | Standard Error |
|---|---|---|---|---|
| Normal | 24 | 0-545 | 90.3 | 71.6 |
| ALS | 92 | 0-1319 | 352.9 | 36.6 |
| Alzheimer | 36 | 38-1457 | 585.6 | 58.5 |
| Parkinson | 26 | 41-952 | 629.2 | 68.8 |

Protein 2307 Concentrations in the Diagnosis, Prognosis and Therapeutics of Neurodegenerative Disease

As shown in Table 2, normal subjects have very low values of protein 2307. Although the ALS, AD and PD patients exhibited a wide range of protein 2307 concentrations, it is apparent that a very low value of protein 2307 concentration suggests that a patient does not have AD or PD. For example, a concentration of protein 2307 that was less than or equal to 100 ppm was present in 16 of 24 (66.7%) of normal subjects, 24 of 92 (26.1%) of the ALS patients, 5 of 36 (13.9%) of the AD patients, and 4 of 26 (15.4%) of the PD patients. Thus, a value of less than 100 ppm of protein 2307 supports that a patient does not have neurodegenerative disease, especially not AD or PD.

In contrast, a very high value of protein 2307 is an indicator of neurodegenerative disease. For example, a value of 300 ppm or more of protein 2307 was present in only 3 of the 24 normal subjects (12.5%), 40 of the 92 ALS patients (43.4%), 24 of the 36 AD patients (66.7%), and 21 of 26 PD patients (80.8%). Thus, a value of 300 ppm or more of protein 2307 strongly suggests that a patient has a neurodegenerative disease. In fact, individuals having a protein 2307 concentration that is greater than or equal to 162 ppm (the mean+1 S.D. of normal values of protein 2307) should consider additional testing.

The test results were subjected to a Bonferroni (pairwise) multiple comparison analysis. The Bonferroni analysis found that normal subjects were significantly differentiated from ALS, AD and PD patients and that ALS patients were significantly differentiated from AD and PD patients based on the level of protein 2307 in a serum sample. However, final differentiation of AD patients from PD patients may require additional testing.

The serum samples may also be subjected to various other techniques known in the art for separating and quantitating proteins. Such techniques include, but are not limited to gel filtration chromatography, ion exchange chromatography, reverse phase chromatography, affinity chromatography (typically in an HPLC or FPLC apparatus), or any of the various centrifugation techniques well known in the art. Certain embodiments would also include a combination of one or more chromatography or centrifugation steps combined via electrospray or nanospray with mass spectrometry or tandem mass spectrometry of the proteins themselves, or of a total digest of the protein mixtures. Certain embodiments may also include surface enhanced laser desorption mass spectrometry or tandem mass spectrometry, or any protein separation technique that determines the pattern of proteins in the mixture either as a one-dimensional, two-dimensional, three-dimensional or multi-dimensional protein pattern, and/or the pattern of protein post synthetic modification isoforms.

The quantitation of a protein by antibodies directed against that protein are well known in the field. The techniques and methodologies for the production of one or more antibodies to the tripartite motif protein TRIM5 isoform gamma and/or its related peptides are routine in the field and are not described in detail herein.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of human, murine, monkey, rat, hamster, rabbit and even chicken origin. Due to the ease of preparation and ready availability of reagents, murine monoclonal antibodies are generally preferred. However, “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has an antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies to the tripartite motif protein TRIM5 isoform gamma and related peptides may be used in a variety of assays in order to quantitate the protein in serum samples, or other fluid or tissue samples. Well known methods include immunoprecipitation, antibody sandwich assays, ELISA and affinity chromatography methods that include antibodies bound to a solid support. Such methods also include microarrays of antibodies or proteins contained on a glass slide or a silicon chip, for example.

It is contemplated that arrays of antibodies to protein 2307, or peptides derived from protein 2307, may be produced in an array and contacted with the serum samples or protein fractions of serum samples in order to quantitate the tripartite motif protein TRIM5 isoform gamma related peptides. The use of such microarrays is well known in the art and is described, for example in U.S. Pat. No. 5,143,854, incorporated herein by reference.

The present invention includes a screening assay for neurodegenerative disease based on the up-regulation of protein 2307 expression. One embodiment of the assay will be constructed with antibodies to protein 2307 and/or its related peptides. One or more antibodies targeted to antigenic determinants of the tripartite motif protein TRIM5 isoform gamma related protein 2307 will be spotted onto a surface, such as a polyvinyl membrane or glass slide. As the antibodies used will each recognize an antigenic determinant of protein 2307, incubation of the spots with patient samples will permit attachment of the protein 2307 and its related peptides to the antibody.

The binding of protein 2307 and its related peptides can be reported using any of the known reporter techniques including radioimunoassays (RIA), stains, enzyme-linked immunosorbant assays (ELISA), sandwich ELISAs with a horseradish peroxidase (HRP)-conjugated second antibody also recognizing the protein 2307, the pre-binding of fluorescent dyes to the proteins in the sample, or biotinylating the proteins in the sample and using an HRP-bound streptavidin reporter. The HRP can be developed with a chemiluminescent, fluorescent, or colorimetric reporter. Other enzymes, such as luciferase or glucose oxidase, or any enzyme that can be used to develop light or color can be utilized at this step.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A biomarker of neurodegenerative disease comprising an increased quantity of a tripartite motif protein TRIM5 isoform gamma related peptide in a serum sample.

2. The biomarker of claim 1, wherein the neurodegenerative disease is ALS.

3. The biomarker of claim 1, wherein the neurodegenerative disease is Parkinson's disease.

4. The biomarker of claim 1, wherein the neurodegenerative disease is Alzheimer's disease.

5. The biomarker of claim 1, wherein the tripartite motif protein TRIM5 isoform gamma related peptide includes an antigenic determinant of the tripartite motif protein TRIM5 isoform gamma.

6. The biomarker of claim 1, wherein the tripartite motif protein TRIM5 isoform gamma related peptide has the amino acid sequence of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, or a combination thereof.

7. A method for screening for neurodegenerative disease comprising:
obtaining a serum sample from a test subject;
determining a quantity of at least one tripartite motif protein TRIM5 isoform gamma related peptide in the serum sample; and
comparing the quantity of the tripartite motif protein TRIM5 isoform gamma related peptide in the test subject serum sample with a range of normal values of the tripartite motif protein TRIM5 isoform gamma related peptide in control subjects;
whereby an increase in the quantity of the tripartite motif protein TRIM5 isoform gamma related protein in the serum sample to a level greater than the range of normal values of tripartite motif protein TRIM5 isoform gamma related peptide is indicative of a neurodegenerative condition.

8. The method of claim 7, wherein the neurodegenerative condition is ALS.

9. The method of claim 7, wherein the neurodegenerative condition is Parkinson's disease.

10. The method of claim 7, wherein the neurodegenerative condition is Alzheimer's disease.

11. The method of claim 7, wherein the tripartite motif protein TRIM5 isoform gamma related peptide includes an antigenic determinant located within the amino acid sequence of SEQ ID No. 1.

12. The method of claim 7, wherein the tripartite motif protein TRIM5 isoform gamma related peptide has the amino acid sequence of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26,or a combination thereof.

13. The method of claim 7, wherein the upper range of normal values of the tripartite motif protein TRIM5 isoform gamma related peptide in neurodegenerative disease is equal to a 95% upper confidence limit in a concentration of the tripartite motif protein TRIM5 isoform gamma related peptide determined in a set of serum samples collected from control subjects free of the neurodegenerative condition.

14. A method of diagnosing a neurodegenerative disease, the method comprising:
collecting a serum sample from a test subject;
analyzing the serum sample for an increased expression of tripartite motif protein TRIM5 isoform gamma related protein; and
using the expression of tripartite motif protein TRIM5 isoform gamma related protein to diagnose the test subject.

15. The method of claim 14, wherein the diagnosis is an adjunct to at least one other diagnostic test for the neurodegenerative disease.

16. The method of claim 14, wherein the expression of the tripartite motif protein TRIM5 isoform gamma related protein is determined using two-dimensional gel electrophoresis.

17. The method of claim 16, wherein the two-dimensional gel electrophoresis comprises a separation by isoelectric point followed by a separation by molecular weight.

18. The method of claim 16, wherein the two-dimensional gel is stained and an intensity of the tripartite motif protein TRIM5 isoform gamma related protein staining is proportional to the expression of the tripartite motif protein TRIM5 isoform gamma related protein in the serum sample.

19. A method for diagnosing neurodegenerative disease comprising:
obtaining a serum sample from a patient and a set of control serum samples;
determining a quantity of a tripartite motif protein TRIM5 isoform gamma related peptide in the patient serum sample and the set of control samples; and
comparing the quantity of the tripartite motif protein TRIM5 isoform gamma related protein in the patient serum with the quantity of the tripartite motif protein TRIM5 isoform gamma related peptide in the set of control samples to diagnose a neurodegenerative condition.

20. The method of claim 19, wherein the quantity of the tripartite motif protein TRIM5 isoform gamma related peptide is determined using an antibody directed against an antigenic determinant in the tripartite motif protein TRIM5 isoform gamma.

21. The method of claim 19, wherein the quantity of the tripartite motif protein TRIM5 isoform gamma related peptide is determined by contacting the serum with at least one antibody with reactivity to the amino acid sequence of SEQ ID No. 1.

22. The method of claim 19, wherein the quantity of the tripartite motif protein TRIM5 isoform gamma related peptide is determined using two-dimensional gel electrophoresis.

23. The method of claim 22, wherein the two-dimensional gel electrophoresis comprises a separation by isoelectric point followed by a separation by molecular weight.

24. The method of claim 19, wherein the quantity of the tripartite motif protein TRIM5 isoform gamma related peptide is determined by contacting the serum with at least one antibody with reactivity to the amino acid sequence of SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, or a combination thereof.

25. A method for diagnosing neurodegenerative disease comprising:
obtaining a patient serum sample;
determining a protein expression pattern of the serum sample by two-dimensional gel electrophoresis;
quantitating a tripartite motif protein TRIM5 isoform gamma protein related protein in the protein expression pattern; and
using the quantity of the tripartite motif protein TRIM5 isoform gamma related protein to diagnose a neurodegenerative condition.

26. The method of claim 25, further comprising performing an additional diagnostic test for the neurodegenerative condition.

27. The method of claim 25, wherein the two-dimensional gel electrophoresis comprises a separation by isoelectric point followed by a separation by molecular weight.

28. The method of claim 25, wherein the quantity of tripartite motif protein TRIM5 isoform gamma related protein is determined using an antibody directed against an antigenic determinant in the tripartite motif protein TRIM5 isoform gamma protein.

29. The method of claim 25, wherein the quantity of tripartite motif protein TRIM5 isoform gamma protein in the patient serum sample is determined by contacting the two-dimensional gel with at least one antibody with reactivity to the tripartite motif protein TRIM5 isoform gamma related protein.

30. The method of claim 25, wherein a plurality of antibodies reactive with an antigenic determinant in the tripartite motif protein TRIM5 isoform gamma protein are used to determine the quantity of the tripartite motif protein TRIM5 isoform gamma related protein in the patient serum sample.

31. The method of claim 25, wherein the antibody is a monoclonal antibody.

32. The method of claim 25, wherein the antibody is a chimeric antibody.

33. The method of claim 25, wherein the antibody is an antiserum, an Fab antibody fragment, a monoclonal antibody, a chimeric antibody, a IgG immunogobulin, an IgM immunoglobulin, or a combination of the same.

34. The method of claim 25, wherein the amount of antibody reacted with the tripartite motif protein TRIM5 isoform gamma related protein is reported using a radioimmunoassay, an enzyme-linked immunosorbent assay, or a sandwich enzyme-linked immunosorbent assay.

35. The method of claim 25, wherein the amount of antibody reacted with the tripartite motif protein TRIM5 isoform gamma related protein is reported using a horse radish peroxidase reporter, a strepavidin reporter, a fluorescent reporter, a chemiluminescent reporter, a calorimetric reporter, or a combination of the same.
